(19) United States
(12) Patent Application Publication
(10) Pub. No.: US 2015/0044679 A1
(43) Pub. Date: Feb. 12, 2015

(54) SYSTEMS, DEVICES, AND METHODS FOR DEPLOYING ONBOARD REAGENTS IN A DIAGNOSTIC DEVICE

(71) Applicant: Xagenic Inc., Toronto (CA)

(72) Inventors: Graham D. Jack, Toronto (CA); Susan Bortolin, Oakville (CA)

(21) Appl. No.: 14/454,635

(22) Filed: Aug. 7, 2014

Related U.S. Application Data
(60) Provisional application No. 61/863,401, filed on Aug. 7, 2013.

(51) Int. Cl. C12Q 1/68

(52) U.S. Cl. CPC C12Q 1/6806
USPC 435/6.11; 435/306.1; 435/287.2

(57) ABSTRACT

Disclosed herein are systems, devices, and methods for detecting the presence of a pathogen in a biological host, such as in a point of care setting. In certain aspects, materials and methods improve point of care devices by providing pre-loaded, preferably dried, agents for performing one or more of sample lysis and signal enhancement inside the device.

### CROSS-REFERENCE TO RELATED APPLICATION

This application claims priority to U.S. Provisional Application No. 61/863,401 filed Aug. 7, 2013, which is hereby incorporated by reference herein in its entirety.

### BACKGROUND

Diagnostic tests for various diseases can provide important information for successful treatment. Diagnostic assays are used to detect pathogens, including bacteria and viruses. Many standard diagnostic assays, such as cell cultures and genetic testing with PCR amplification, require sending samples to labs and have long turnaround times of several days or weeks. Many patients, in such cases, do not return to the care provider to receive the results or treatments, and in some cases, the long turn-around can compromise the ability to properly treat the condition.

While some assays have been automated, many still require significant expertise or training. In many currently available systems the cells to be tested are not adequately processed prior to applying the tests, which can introduce inaccuracies. Alternative systems and methods for diagnostics, could be beneficial for improved patient outcomes, particularly in point of care applications.

### SUMMARY

This application is directed to systems, devices and methods for preparing materials and samples to be used within a point of care device to improve its use in detecting target molecules within a patient's sample. In general, the systems, devices and methods relate to approaches to integrating agents and materials that can be used to prepare samples and react with the samples to detect target molecule. To provide an overall understanding of the systems, devices, and methods described herein, certain illustrative embodiments will be described. It is to be understood that the systems, devices, and methods disclosed herein, while shown for use in diagnostic systems for bacterial diseases such as Chlamydia, may be applied in other applications including, but not limited to, detection of other bacteria, viruses, fungi, prions, plant matter, animal matter, protein, RNA sequences, DNA sequences, as well as cancer screening and genetic testing, including screening for genetic traits and disorders.

Disclosed herein are systems, devices, and methods for detecting the presence of a pathogen in a biological host, such as in a point of care setting. In certain aspects, materials and methods improve point of care devices by providing pre-loaded, preferably dried, agents for performing one or more of sample lysis and signal enhancement inside the device.

The systems, devices, and methods described herein may be used for diagnosing a disease in a living organisms such as a human or animal. For example, Chlamydia is a bacterial disease that afflicts humans and is caused by the bacteria Chlamydia trachomatis. A caretaker, such as a nurse or physician, may obtain a sample from a patient desiring to receive a diagnosis for this disorder. For example, the caretaker may use a medical swab to wipe the surface of the vagina, to thereby obtain a biological sample of vaginal fluid and vaginal epithelial cells. If the patient is carrying the Chlamydia trachomatis bacteria, the bacteria would be present in the sample. Additional markers specific to the human genome would also be present. The caretaker or technician then uses the systems, devices, and methods described herein to detect the presence or absence of the bacteria or other pathogen, cell, protein, or gene in the sample.

In general, the diagnostic systems disclosed herein use probe molecules, preferably protein nucleic acid probes, to detect components within a sample that have matching genetic sequences to the nucleotide sequences of the probe. In that way, bacteria or virus other components of the sample can be detected. Under appropriate conditions, the probe can hybridize to a complementary target marker in the sample to provide an indication of the presence of target marker in the sample. In certain approaches, the sample is a biological sample from a biological host. For example, a sample may be tissue, cells, proteins, fluid, genetic material, bacterial matter or viral matter a plant, animal, cell culture, or other organism or host. The sample may be a whole organism or a subset of its tissues, cells or component parts, and may include cellular or non-cellular biological material. Fluids and tissues may include, but are not limited to, blood, plasma, serum, cerebrospinal fluid, lymph, tears, saliva, blood, mucus, lymphatic fluid, synovial fluid, cerebrospinal fluid, amniotic fluid, amniotic cord blood, urine, vaginal fluid, semen, tears, milk, and tissue sections. The sample may contain nucleic acids, such as deoxyribonucleic acids (DNA), ribonucleic acids (RNA), or copolymers of deoxyribonucleic acids and ribonucleic acids or combinations thereof. In certain approaches, the target marker is a nucleic acid sequence that is known to be unique to the host, pathogen, disease, or trait, and the probe provides a complementary sequence to the sequence of the target marker to allow for detection of the host sequence in the sample. Examples of probes and their use in electrochemical detection assays are disclosed in in further detail in U.S. Pat. Nos. 7,361,470 and 7,741,033, and PCT Application No. PCT/US12/024,015, and U.S. Provisional Application No. 61/700,285, which are hereby incorporated by reference herein in their entireties.

In certain aspects, systems, devices and methods are provided to perform processing steps, such as purification and extraction and signal amplification, on the sample. Analytes or target molecules for detection, such as nucleic acids, are sequestered inside of cells, bacteria, or viruses. The sample is processed to separate, isolate, or otherwise make accessible, various components, tissues, cells, fractions, and molecules included in the sample. Processing steps may include, but are not limited to, purification, homogenization, lysing, and extraction steps, as well as signal amplification. The processing steps may separate, isolate, or otherwise make accessible a target marker, such as the target marker in or from the sample, and they may also or in addition help amplify the signal detected by the diagnostic system.

In certain approaches, the target marker is genetic material in the form of DNA or RNA obtained from any naturally occurring prokaryotes such, pathogenic or non-pathogenic bacteria (e.g., Escherichia, Salmonella, Clostridium, Chlamydia, etc.), eukaryotes (e.g., protozoans, parasites, fungi, and yeast), viruses (e.g., Herpes viruses, HIV, influenza virus, Epstein-Barr virus, hepatitis B virus, etc.), plants, insects, and animals, including humans and cells in tissue culture. Target nucleic acids from these sources may, for example, be found in biological samples of a bodily fluid from an animal, including a human. In certain approaches, the sample is obtained from a biological host, such as a human patient, and includes non-human material or organisms, such as bacteria, viruses, other pathogens.

In one aspect, a biological sample is processed to release or otherwise make accessible, the target molecules or analytes of interest, such as the target marker and control marker. For example, analytes, such as nucleic acids, may normally be sequestered inside of cells, bacteria, or viruses from which they need to be released prior to characterization. For example, mechanical approaches including, but not limited to, sonication, centrifugation, shear forces, heat, and agitation may be used to process a biological sample. Additionally or alternatively, chemical methods including, but not limited to, surfactants, chaotropes, enzymes, or heat may be applied to produce a chemical effect.

U.S. Application No. 61/700,285 describes diagnostic devices and systems that include an on-board lysis chamber for applying lysis techniques to a biological sample to release target markers from cells within the sample, prior to analyzing the contents of the sample. The contents of that application are hereby incorporated by reference. Lysis techniques disrupt the integrity of a biological compartment such as a cell such that internal components, such as RNA, are exposed to and may enter the external environment. Lysis procedures may cause the formation of permanent or temporary openings in a cell membrane or complete disruption of the cell membrane, to release cell contents into the surrounding solution. For example, a modulated electrical potential can be applied to a sample to release nucleic acids, and in particular, RNA, into the sample solution. Electrical lysis techniques are described in further detail in PCT application No. PCT/US12/28721, the contents of which are hereby incorporated herein by reference. The device and systems of those earlier filed applications can also be modified to include a lysing chamber that uses a chemical lysing agent on board the device. A brief description of these techniques, as applied to the current system, is provided below.

### BRIEF DESCRIPTION OF THE DRAWINGS

The foregoing and other objects and advantages will be appreciated more fully from the following further description thereof, with reference to the accompanying drawings. These depicted embodiments are to be understood to as illustrative and not as limiting in any way:

FIG. 1 depicts a lysis chamber that is configured to be integrated within a point of care device

FIG. 2 depicts a system for preparing and analyzing a biological sample that can be configured within a point of care device.

FIG. 3A-FIG. 4 depict embodiments of an on-board lysing chamber structured to lyse biological samples using chemical lysing agents and which can be integrated into the system of FIG. 2.

FIG. 5A depicts a cartridge system for receiving, preparing, and analyzing a biological sample.

FIG. 5B depicts an embodiment of a cartridge for an analytical detection system.

FIG. 6 depicts an automated testing system to provide ease of processing and analyzing a sample.

FIG. 7 depicts a hand-held point of care device.

FIG. 8 depicts in further detail components of this hand-held system illustrated in FIG. 8.

FIGS. 9A-9E depict the use and operation of the system or the hand-held device illustrated in FIG. 8.

FIG. 10 illustrates an example performed using the system.

### DETAILED DESCRIPTION

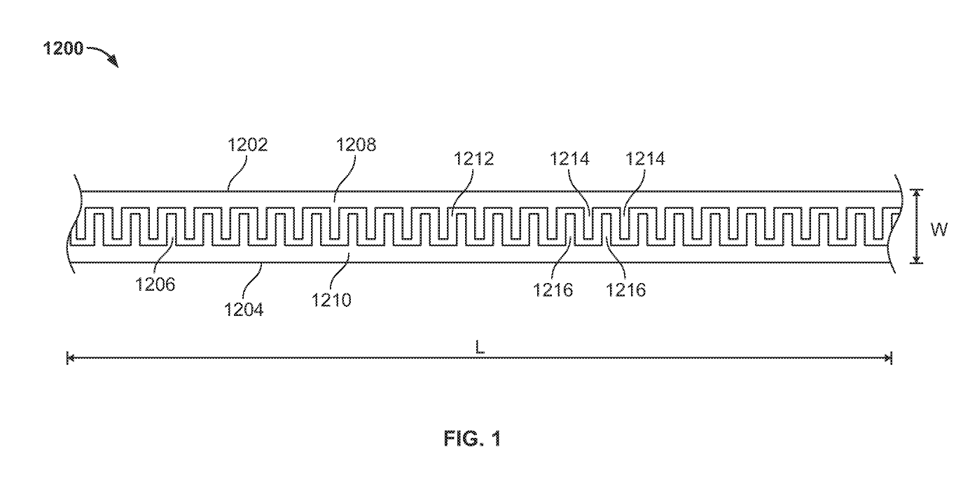

FIG. 1 depicts a lysis chamber that is configured to be integrated within a point of care device. The example shown in FIG. 1 is an electrical lysis chamber but as discussed below, can be modified to provide a chemical lysis chamber on-board the device. Chamber 1200 includes a first wall 1202 and a second wall 1204 defining a space 1206 in which a sample is retained. For example, a sample may flow through the space 1206 of the lysis chamber 1200. Chamber 1200 also includes at least one lysing source (as shown, two lysing sources are included—a first electrode 1208 and second electrode 1210). First lysing source (1208) and second lysing source (1210) are separated by a spacing 1212.

First source 1208 and second source 1210 may be electrical or chemical lysing sources. For example, electrodes may be used that are composed of a conductive material. For example, first source 1208 and second source 1210 may comprise carbon or metal electrodes including, but not limited to, gold, silver, platinum, palladium, copper, nickel, aluminum, ruthenium, and alloys. First source 1208 and second source 1210 may comprise conductive polymers, including, but not limited to polypyrole, iodine-doped transpolyacetylene, poly(dioctyl-bithiophene), polyaniline, metal impregnated polymers and fluoropolymers, carbon impregnated polymers and fluoropolymers, and admixtures thereof. In certain embodiments, first source 1208 and second source 1210 comprise a combination of these materials.

In certain embodiments, the spacing 1212 separates the first source 1208 and the second electrode 1210 by a range of approximately 1 nm to approximately 2 mm. In certain embodiments, the first electrode 1208 and the second electrode 1210 are inter-digitated electrodes. For example, the first electrode 1208 may have digits 1214 spaced between digits 1216 of the second electrode 1210. The spacing 1212 can be composed of an insulating material to further localize the applied potential difference to the electrodes. For example, spacing 1212 may comprise silicon dioxide, silicon nitride, nitrogen doped silicon oxide (SiOxNy), paralyene, or other insulating or dielectric materials.

In the example of FIG. 1, first source 1208 and second source 1210 are planar electrodes, over which the sample flows. For example, first electrode 1208, second electrode 1210, and spacing 1212 are coplanar to form a base within space 1206 of the chamber 1200. First electrode 1208 and second electrode 1210 may also comprise other configurations, including, but not limited to, arrays, ridges, tubes, and rails. First source 1208 and second source 1210 may be positioned on any portion of chamber 1200, including, but not limited to sides, bottom surfaces, upper surfaces, and ends. The lysis chamber 1200, first source 1208, second source 1210, and spacing 1212 may have any appropriate length L. Although depicted as having the same length L in FIG. 12, each component of the chamber 1200 may have a different length. In certain approaches, the length L of the chamber 1200 is between approximately 0.1 mm and 100 mm. For example, the chamber 1200 may have a length L of approximately 50 mm. Similarly, the lysis chamber 1200, first source 1208, second source 1210, and spacing 1212 may have any appropriate width W. Each component of the chamber may have a different width. In certain approaches, the width w of the chamber 1200 is between approximately 0.1 mm and 10 mm. For example, the chamber 1200 may have a width W of 2 mm. The chamber 1200 is depicted as linear or straight, however, in certain approaches, the chamber 1200 includes turns, bends, and other nonlinear structures.

In certain approaches, lysing pulses (either electrical by electrical pulses or chemical, e.g., by depositing aliquots of chemical lysing agents into the lysing chamber) are applied as the sample continuously flows through chamber 1200. Lysis pulses may also be applied while the sample is immobile in the chamber, or during agitation of the sample. In embodiments using electrical lysis, the total application time of the pulses is between about 1 second and 1000 seconds. In certain approaches, the pulses are applied for about 2-3 minutes. In certain approaches, the pulses are applied for about 20 seconds or less.

In certain embodiments, the lysis procedure controllably fragments analyte molecules, such as DNA and RNA. Fragmentation can advantageously reduce the time required to detect or otherwise characterize the released analyte. For example, fragmentation of an analyte molecule may reduce molecular weight and increase speed of diffusion, thereby enhancing molecular collision and reaction rates. In another example, fragmenting a nucleic acid may reduce the degree of secondary structure, thereby enhancing the rate of hybridization to a complementary probe molecule. For example, RNA from a cell lysed by the application of a modulated potential to first electrode 1208 and second electrode 1210 may have an average length of over 2,000 bases immediately upon lysis, but are rapidly cleaved into fragments of reduced length under continued lysing conditions. The average size of such fragments may be up to about between about 20% and about 75% of the size or length of the unfragmented analyte. In certain approaches, the analyte is a RNA. For example, fragmented RNA may have a significant portion of molecules with lengths between approximately 20 and approximately 500 base pairs. In certain approaches, pulses are modulated to simultaneously lyse and fragment the sample and analytes. Additionally or alternatively, a second set of lysing (e.g., electrical or chemical) pulses may be applied and configured to provide specific, controlled fragmentation. For example, a first set of pulses may applied to provide lysis, and a second set of pulses may be applied to provide fragmentation. In certain approaches, the first pulse set for lysis and second pulse set for fragmentation are alternated.

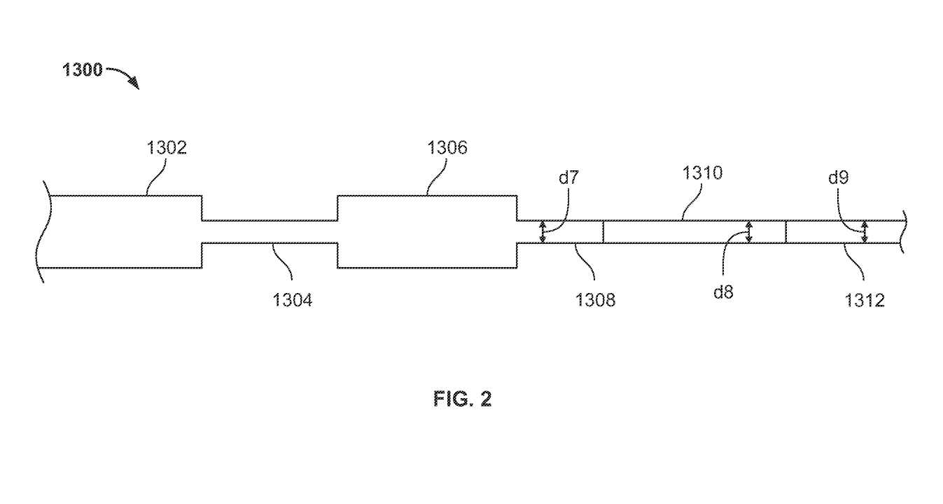

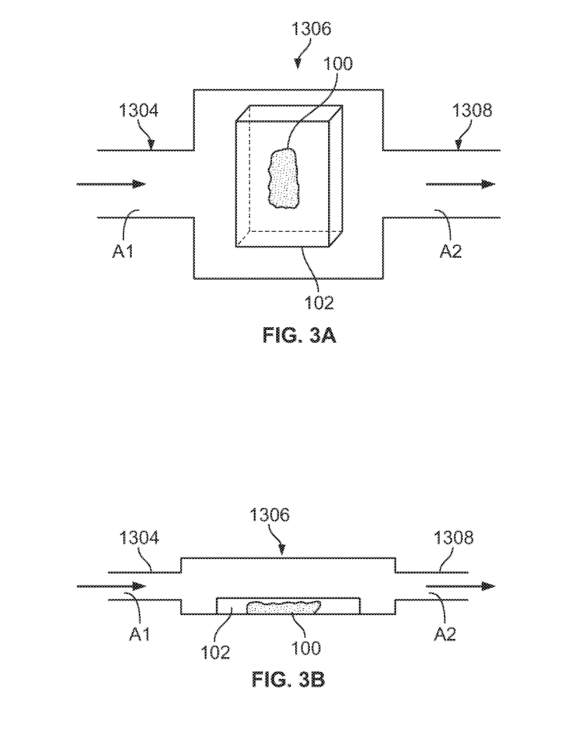

FIG. 2 depicts a system for preparing and analyzing a biological sample that can be configured within a point of care device. System 1300 includes a receiving chamber 1302, a first channel, 1304, a lysis chamber 1306, a second channel 1308, an analysis chamber 1310, and a third channel 1312. Other processing chambers and channels may also be included. In practice, a user obtains a sample from a biological host and places the sample in receiving chamber 1302. While in receiving chamber 1302, the sample may undergo processing, such as filtering to remove undesirable matter, addition of reagents, and removal of gases. The sample is then moved from receiving chamber 1302 through channel 1304 and into lysis chamber 1306. The sample may be moved by applying external pressure with fluids or gases, for example, with a pump or pressurized gas. In certain embodiments, lysis chamber 1306 is similar to lysis chamber 1200 of FIG. 1 and can be configured with electrical lysing agents such as electrodes. In other embodiments the lysis chamber 1306 is configured as a receptacle that contains one or more lysing chemical agents (as exemplified in FIGS. 3A-10 below). Inside the chamber 1306, the sample undergoes a lysis procedure, such as an electrical or chemical lysis procedure that lyses the cells in the sample to release the analytes contained therein, including genetic material. The lysis procedure may also cause fragmentation of the analytes released from the cells, such as RNA, which serve as target markers and control marker.

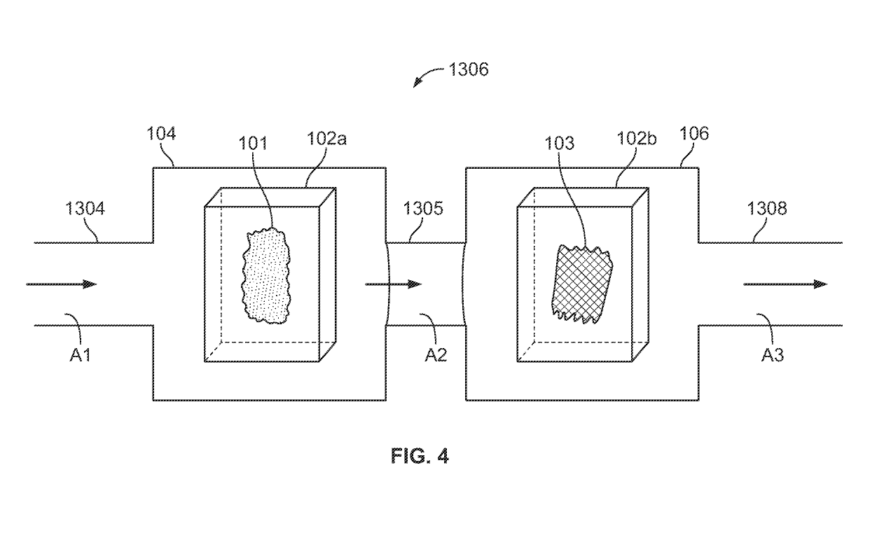

FIG. 3A-FIG. 4 depict embodiments of an on-board lysing chamber 1306 structured to lyse biological samples using chemical lysing agents and which can be integrated into the system of FIG. 2. FIG. 3A depicts the chamber 1306 with inlet channel 1304 and outlet channel 1308, as per FIG. 2. Inside chamber 1306 is a compartment 102 that contains a chemical lysing agent 100. Preferably, the lysing agent 100 is in solid, dried form within the compartment 102. In use, a sample to be tested flows into the chamber 1306 via inlet line 1304 (depicted as arrow A1) and while inside the chamber 1306 flows into the compartment 102, whereupon the liquid sample inlet mixes with and dissolves the lysing agent 100. For example, the inlet sample could be a sample buffer containing bacteria or virus that the system is intended to analyze. That buffer, upon contacting the agent 100 within the chamber 102, then dissolves the agent 100, changes the pH of the sample which starts a lysing reaction that chemically lyses the cells within the sample. Lysing the cells also exposes the cellular analytes and other components to the lysing agent, which fragments and denatures the components. Included among those components, the genetic material from the cell will fragment when contacting that lysing agent, creating smaller fragments that can more readily bind to probe sequences and are more readily detectable by the diagnostic system contained in the analysis chamber 1310 of FIG. 2. To that end, lysis exposure time is preferably controlled so that the nucleic acids in the sample are partially fragmented within the sample by the changed pH. The sample, after mixing and at least partial dissolution with the lysing agent, then exits the chamber 1306 via outlet 1308 (as depicted by arrow A2).

FIG. 4 depicts an alternative embodiment of lysing chamber 1306. As shown, the chamber 1306 includes two chambers 104 and 106. Chamber 104 includes compartment 102a that has lysing agent 101, for example, a strong base such as NaOH that can lyse cells and denature and fragment genetic and biologic materials in a sample. The lysing reaction that occurs within the compartment 102a (which is similar to the compartment 102 of FIG. 3A) is preferably quenched after a certain period of time to stop the lysis of the materials, leaving them in fragmented form so as to prevent ultimate destruction and degradation of the materials beyond their usability in the detection system. Accordingly, second chamber 106 includes a second compartment 102b that houses a neutralizing agent 103. For example, this neutralizing agent could be a strong acid that lowers the pH of the sample after it is lysed by the base 101, to thereby prevent further degradation and denaturation of the genetic material in the sample. In use, the sample flows into the chamber 1306 via inlet line 1304 (see arrow A1) and undergoes lysis and denaturation of its contents within the first chamber 104, and after which it flows into the second chamber 106 via intermediate line 1305 (arrow A2), whereupon the reaction is quenched. The resulting sample flows out of the chamber 1306 via outlet line 1304 (see arrow A3).

The lysis chambers of FIGS. 2-4 allow lysis of target sample cells (e.g., virus or bacteria) to be performed on-board the device, preferably by a strong chemical agent (e.g., a base, such as NaOH). A detergent (e.g., SDS, tween, tritonX) is preferably also used in combination with the chemical agent (e.g., the base in the lysing chamber 104). In certain implementations, a base is selected as the chemical agent and deposited by drying it to the interior walls of the compartment 102a inside the lysis chamber 104. In one mode during lysis, hydroxide from the strong base attacks and breaks down the cells inside compartment 102a and allows the detergent to create holes in the cellular membrane, thus lysing the bacteria and releasing its genetic material (DNA, RNA) into solution. The released material is then at least partially fragmented by the hydroxide solution. This reaction can then be neutralized in compartment 102b with the addition of a strong acid to prevent further degradation/denaturation of the genetic material. In certain implementations this lysis process is performed within a single use, hand-held cartridge containing fully active, dried down, long-term room temperature stable reagents.

In one advantage, the on-board lysing approach also helps stabilize the lysis agent. Many acids are easily dried down and maintain full activity. However, challenges exist in drying down NaOH and maintaining its activity over a period of time. NaOH in its dry form rapidly takes on moisture from its environment and allows dissolved CO₂ to change the base into sodium bicarbonate. This is potentially problematic when drying down liquid NaOH as dissolved CO₂ concentrates in the liquid. The approach described herein provides an elegant solution to that problem, allowing the base to be stabilized for longer term storage or use.

In the point of case implementation, to prepare the cartridge, the lysing agent(s) are actively dried onto a surface within the interior of the chamber 1306. In the case of FIG. 4, active spots of both base and acid are dried on the floor of the separate compartments (102a and 102b) of the cartridge. For example, dry powder NaOH and Citric Acid are dissolved in a degassed DiH₂O, forming two different liquids, thus preventing NaOH exposure to any dissolved CO₂. These two liquids are then spotted (in μl volumes) in the separate compartments 102a and 102b of the cartridge. These spots are rapidly dried down in a vacuum oven, limiting exposure to air and reactive CO₂. In certain implementations, the cartridge may optimally be quickly packaged into nitrogen purged moisture barrier bags preventing further exposure to moisture and CO₂. These procedures and conditions allow for the activity of NaOH to remain stable under long-term, room temperature environments.

Using dry lysis reagents in separate chambers allows the use of a neutral pH sample buffer (e.g., containing a detergent) to flow the sample through the system. The buffer (e.g., phosphate buffered saline solution) carries the sample into the chamber 102a containing the dry NaOH spot. As the sample buffer containing bacteria flows into the chamber, the buffer dissolves the NaOH spot, raising the pH of the buffer which causes the cells in the sample to lyse. As explained further below, after lysis in chamber 102a, the sample fluid is then pushed into the compartment 102b containing the dry acid spot 103. The acid spot 103 is dissolved and mixed as the solution enters the compartment 102b via fluid line 1305 (arrow A2). This lowers the pH of the buffer, neutralizing it, and prevents further degradation of the genetic material. The sample, in the neutralized buffer, is then sent to the analysis chamber 1310 (described below) through channel 1308. Analysis chamber 1310 may include any of analysis chambers 400, 500, 600, 700, 800, 900, 1000, and 1100 described in U.S. Provisional Application No. 61/700,285.

The lysing process partially degrades and denatures target genetic material, which helps facilitate direct hybridization detection of nucleic acids of a target when inside the analysis chamber. Smaller fragments of RNA and denatured genomic DNA bind more readily to probe sequences as the secondary structures of these molecules are destroyed. This allows for both increased diffusion of these molecules in solution (increasing hybridization events) and increases accessibility of these to sequences (unfolding) for hybridization. Using separate compartments for base lysis and acid neutralization, the flow from chamber to chamber can be timed (and the on-board fluid pump controlled accordingly) to optimize efficient lysis in concert with adequate degradation/denaturation of genetic material for optimal detection.

Referring back to FIG. 2, the analysis chamber 1310 includes one or more sensors, such as pathogen sensors, host sensors, and non-sense sensors. The target markers and control markers can hybridize with probes on the respective sensors. The presence of the target markers and control markers are analyzed at the sensors, for example, with electrocatalytic techniques, as described previously in relation to FIGS. 1-3. In certain approaches, the sample is then pumped through channel 1312 to additional processing, storage, or waste areas. Further examples of sensor structures and applications are disclosed in U.S. Provisional Application No. 61/700,285, incorporated by reference herein.

The dimensions, such as lengths, widths, and diameters of the sections of system 1300 can be configured to adjust for different volumes, flow rates, or other parameters. FIG. 2 depicts channel 1308 with diameter d7, analysis chamber 1310 with diameter d8, and channel 1312 with diameter d9. In certain approaches, diameters d7, d8, and d9 are each approximately the same to provide an even flow into and through analysis chamber 1310. In certain approaches, diameters d7, d8, and d9 have different sizes to accommodate for different flow rates, the addition of reagents, or removal of portions of the sample.

In certain approaches, the systems, devices, and methods described herein are used for diagnosing a disease in a human. The systems, devices, and methods may be used to detect bacteria, viruses, fungi, prions, plant matter, animal matter, protein, RNA sequences, DNA sequences, cancer, genetic disorders, and genetic traits. For example, the disorder Chlamydia is a bacterial disease caused by the bacteria Chlamydia trachomatis. A caretaker, such as a nurse or physician, may obtain a sample from a patient desiring to receive a diagnosis for this disorder. For example, the caretaker may use a medical swab to wipe a surface of the vagina, to thereby obtain a biological sample of vaginal fluid and vaginal epithelial cells. If the patient is carrying the Chlamydia trachomatis bacteria, the bacteria would be present in the sample. Additionally, markers specific to the human genome would also be present. The caretaker or technician may then use the systems, devices, and methods described herein to detect the presence or absence of the bacteria or other pathogen, cell, protein, or gene.

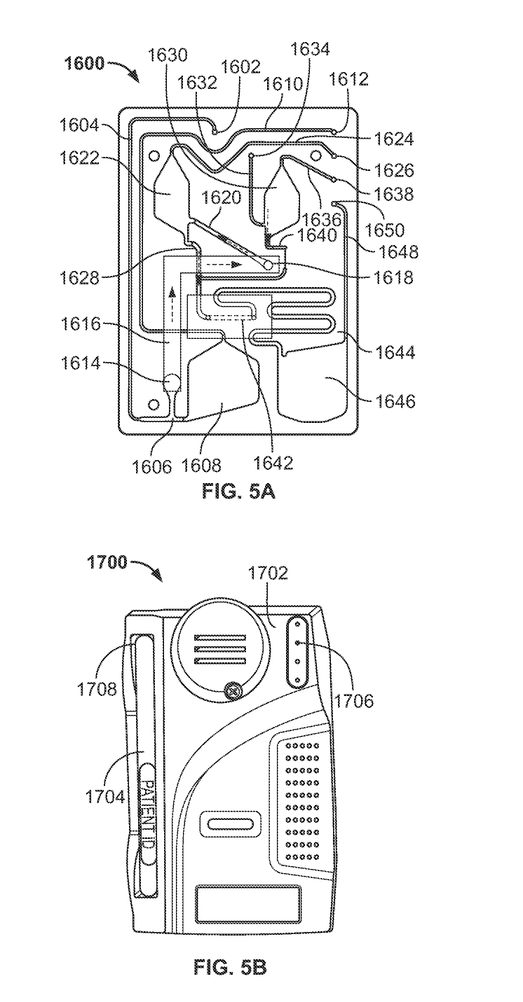

The systems, devices, methods, and electrode and lysis zone embodiments described above may be incorporated into a cartridge to prepare a sample for analysis and perform a detection analysis. FIG. 5A depicts a cartridge system for receiving, preparing, and analyzing a biological sample. For example, cartridge system 1600 may be configured to remove a portion of a biological sample from a sample collector or swab, transport the sample to a lysis zone where a lysis and fragmentation procedure are performed, and transport the sample to an analysis chamber for determining the presence of various markers and to determine a disease state of a biological host.

The system 1600 includes ports, channels, and chambers. System 1600 may transport a sample through the channels and chambers by applying fluid pressure, for example with a pump or pressurized gas or liquids. In certain embodiments, ports 1602, 1612, 1626, 1634, 1638, and 1650 may be opened and closed to direct fluid flow. In use, a sample is collected from a patient and applied to the chamber through port 1602. In certain approaches, the sample is collected into a collection chamber or test tube, which connects to port 1602. In practice, the sample is a fluid, or fluid is added to the sample to form a sample solution. In certain approaches, additional reagents are added to the sample. The sample solution is directed through channel 1604, past sample inlet 1606, and into degassing chamber 1608 by applying fluid pressure to the sample through port 1602 while opening port 1612 and closing ports 1626, 1634, 1638, and 1650. The sample solution enters and collects in degassing chamber 1608. Gas or bubbles from the sample solution also collect in the chamber and are expelled through channel 1610 and port 1612. If bubbles are not removed, they may interfere with processing and analyzing the sample, for example, by blocking flow of the sample solution or preventing the solution from reaching parts of the system, such as a lysis electrode or sensor. In certain embodiments, channel 1610 and port 1612 are elevated higher than degassing chamber 1608 so that the gas rises into channel 1610 as chamber 1608 is filled. In certain approaches, a portion of the sample solution is pumped through channel 1610 and port 1612 to ensure that all gas has been removed.

After degassing, the sample solution is directed into lysis chamber 1616 by closing ports 1602, 1634, 1638, and 1650, opening port 1626, and applying fluid pressure through port 1612. The sample solution flows through inlet 1606 and into lysis chamber 1616. In certain approaches, system 1600 includes a filter 1614. Filter 1614 may be a physical filter, such as a membrane, mesh, or other material to remove materials from the sample solution, such as large pieces of tissue, which could clog the flow of the sample solution through system 1600. Lysis chamber 1616 may be lysis chamber 1200 or lysis chamber 1306 described previously. When the sample is in lysis chamber 1616, a lysis procedure, such as an electrical or chemical lysis procedure as described in the embodiments above, may be applied to release analytes into the sample solution. For example, the lysis procedure may lyse cells to release nucleic acids, proteins, or other molecules which may be used as markers for a pathogen, disease, or host. In certain approaches, the sample solution flows continuously through lysis chamber 1616. Additionally or alternatively, the sample solution may be agitated while in lysis chamber 1616 before, during, or after the lysis procedure. Additionally or alternatively, the sample solution may rest in lysis chamber 1616 before, during, or after the lysis procedure.

Electrical lysis procedures may produce gases (e.g., oxygen, hydrogen), which form bubbles. Bubbles formed from lysis may interfere with other parts of the system. For example, they may block flow of the sample solution or interfere with hybridization and sensing of the marker at the probe and sensor. Accordingly, the sample solution is directed to a degassing chamber or bubble trap 1622. The sample solution is directed from lysis chamber 1616 through opening 1618, through channel 1620, and into bubble trap 1622 by applying fluid pressure to the sample solution through port 1612, while keeping port 1626 open and ports 1602, 1634, 1638, and 1650 closed. Similar to degassing chamber 1608, the sample solution flows into bubble trap 1622 and the gas or bubbles collect and are expelled through channel 1624 and port 1626. For example, channel 1624 and port 1626 may be higher than bubble trap 1622 so that the gas rises into channel 1624 as bubble trap 1622 is filled. In certain approaches, a portion of the sample solution is pumped through channel 1624 and port 1626 to ensure that all gas has been removed.

After removing the bubbles, the sample solution is pumped through channel 1628 and into analysis chamber 1642 by applying fluid pressure through port 1626 while opening port 1650 and closing ports 1602, 1612, 1634, and 1638. Analysis chamber 1642 is similar to previously described analysis chambers, such as chambers 400, 500, 600, 700, 800, 900, 1000, 1100, and 1306. Analysis chamber 1642 includes sensors, such as a pathogen sensor, host sensor, and non-sense sensor as previously described. In certain approaches, the sample solution flows continuously through analysis chamber 1642. Additionally or alternatively, the sample solution may be agitated while in analysis chamber 1642 to improve hybridization of the markers with the probes on the sensors. In certain approaches, system 1600 includes a fluid delay line 1644, which provides a holding space for portions of the sample during hybridization and agitation. In certain approaches, the sample solution sits idle while in analysis chamber 1642 as a delay to allow hybridization.

System 1600 includes a reagent chamber 1630, which holds electrocatalytic reagents, such as transition metal complexes Ru(NH₃)₆³⁺ and Fe(CN)₆³⁻, for amplifying electrochemical signals that arise when markers in the sample solution bind the probe. This amplification is discussed in further detail in U.S. Pat. Nos. 7,361,470 and 7,741,033, and PCT Application No. PCT/US12/024,015, and U.S. Provisional Application No. 61/700,285, which are hereby incorporated by reference herein in their entireties. In certain approaches, the electrocatalytic reagents are stored in dry form with a separate rehydration buffer. For example, the rehydration buffer may be stored in a foil pouch above rehydration chamber 1630. The pouch may be broken or otherwise opened to rehydrate the reagents.

In certain approaches, a rehydration buffer is pumped into rehydration chamber 1630, where it contacts the dried agents. Adding the buffer may introduce bubbles into chamber 1630. Gas or bubbles may be removed from rehydration chamber 1630 by applying fluid pressure through port 1638, while opening port 1634 and closing ports 1602, 1624, 1626, and 1650 so that gas is expelled through channel 1630 and port 1634. Similarly, fluid pressure may be applied through port 1634 while opening port 1638. After the sample solution has had sufficient time to allow the markers to hybridize to sensor probes in the analysis chamber, the hydrated and degassed reagent solution is pumped through channel 1640 and into analysis chamber 1642 by applying fluid pressure through port 1638, while opening port 1650 and closing all other ports. The reagent solution pushes the sample solution out of analysis chamber 1642, through delay line 1644, and into waste chamber 1646 leaving behind only those molecules or markers which have hybridized at the probes of the sensors in analysis chamber 1642. In certain approaches, the sample solution may be removed from the cartridge system 1600 through channel 1648, or otherwise further processed. The reagent solution fills analysis chamber 1642. In certain approaches, the reagent solution is mixed with the sample solution before the sample solution is moved into analysis chamber 1642, or during the flow of the sample solution into analysis chamber 1642. After the reagent solution has been added, an electrocatalytic analysis procedure to detect the presence or absence of markers is performed, for example any of the analysis procedures described or referenced in U.S. Provisional Application No. 61/700,285 or in U.S. Pat. Nos. 7,361,470 and 7,741,033, and PCT Application No. PCT/US12/024,015, may be applied to the solution to detect the presence or absence of target markers in the sample.

FIG. 5B depicts an embodiment of a cartridge for an analytical detection system. Cartridge 1700 includes an outer housing 1702, for retaining a processing and analysis system, such as system 1600. Cartridge 1700 allows the internal processing and analysis system to integrate with other instrumentation. Cartridge 1700 includes a receptacle 1708 for receiving a sample container 1704. A sample is received from a patient, for example, with a swab. The swab is then placed into container 1704. Container 1704 is then positioned within receptacle 1708. Receptacle 1708 retains the container and allows the sample to be processed in the analysis system. In certain approaches, receptacle 1708 couples container 1704 to port 1602 so that the sample can be directed from container 1704 and processed though system 1600. Cartridge 1700 may also include additional features, such as ports 1706, for ease of processing the sample. In certain approaches, ports 1706 correspond to ports of system 1600, such as ports 1602, 1612, 1626, 1634, 1638, and 1650 to open or close to ports or apply pressure for moving the sample through system 1600.

Cartridges may use any appropriate formats, materials, and size scales for sample preparation and sample analysis. In certain approaches, cartridges use microfluidic channels and chambers. In certain approaches, the cartridges use macrofluidic channels and chambers. Cartridges may be single layer devices or multilayer devices. Methods of fabrication include, but are not limited to, photolithography, machining, micromachining, molding, and embossing.

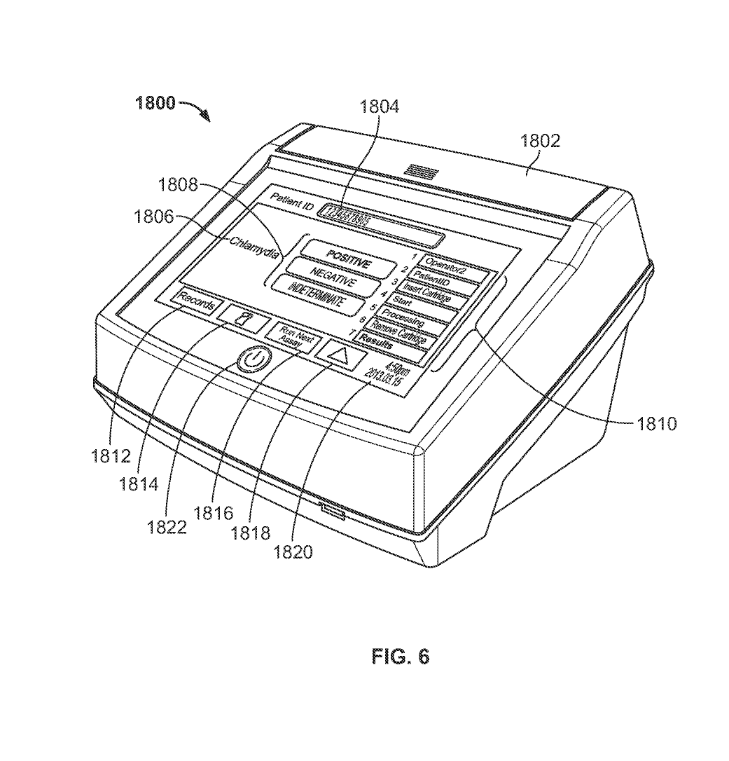

FIG. 6 depicts an automated testing system to provide ease of processing and analyzing a sample. System 1800 may include a cartridge receiver 1802 for receiving a cartridge, such as cartridge 1700. System 1800 may include other buttons, controls, and indicators. For example, indicator 1804 is a patient ID indicator, which may be typed in manually by a user, or read automatically from cartridge 1700 or cartridge container 1704. System 1800 may include a “Records” button 1812 to allow a user to access or record relevant patient record information, “Print” button 1814 to print results, “Run Next Assay” button 1818 to start processing an assay, “Selector” button 1818 to select process steps or otherwise control system 1800, and “Power” button 1822 to turn the system on or off. Other buttons and controls may also be provided to assist in using system 1800. System 1800 may include process indicators 1810 to provide instructions or to indicate progress of the sample analysis. System 1800 includes a test type indicator 1806 and results indicator 1808. For example, system 1800 is currently testing for Chlamydia as shown by indicator 1806, and the test has resulted in a positive result, as shown by indicator 1808. System 1800 may include other indicators as appropriate, such as time and date indicator 1820 to improve system functionality.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in detection systems for bacteria, and specifically, for Chlamydia Trachomatis, may be applied to systems, devices, and methods to be used in other applications including, but not limited to, detection of other bacteria, viruses, fungi, prions, plant matter, animal matter, protein, RNA sequences, DNA sequences, as well as cancer screening and genetic testing, including screening for genetic disorders.

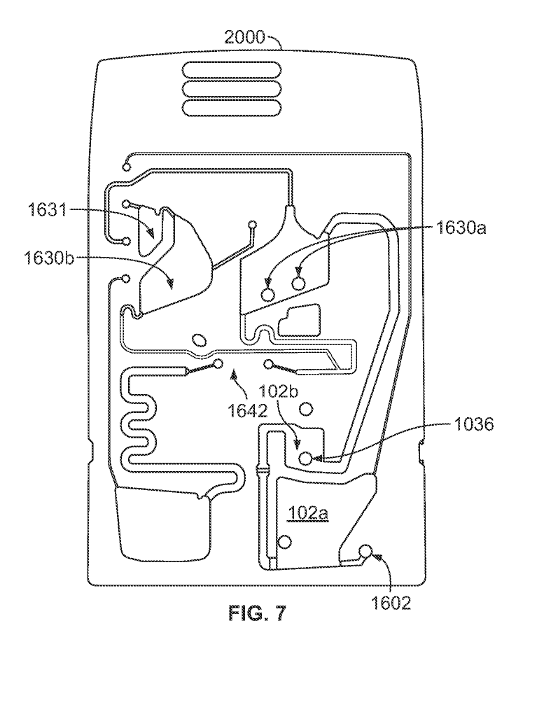

FIGS. 7-9E illustrate an additional embodiment of a point of care device that integrates on-board dried agents that facilitate sample preparation and lysis as well as catalyzing and enhancing the signal in the analysis chamber. The embodiment shown in those figures includes lysis chamber 1306, including the two compartments 102a and 102b discussed above, but it would be understood that the same point of care device could be configured with a single lysis chamber 1306 with a lysing agent such as a chemical lysing agent having a predetermined concentration sufficient to chemically lyse the cells and partially fragment the cell analytes contained in a patient sample that flows therein. In the depicted embodiment, the dual chamber system of FIG. 4 is used. This system is a variation on the system shown in FIGS. 4-6, such that analytical data developed or obtained through the use of the system could be programmed and viewed and manipulated and recorded, printed and otherwise controlled by the testing system shown in FIG. 6.

FIG. 7 depicts a hand-held point of care device 2000 having a sample inlet chamber 1602, a lysing chamber 1306, an analysis chamber with a sensor 1642 that receives fluid from the lysing chamber 1306 after it has been processed through the lysing chamber 1306 and reagent chamber 1630a and 1630b. The reagent chambers 1630a and 1630b perform a similar function and, in example embodiments, identical function as the reagent chamber 1630 in FIGS. 4-5, in that they contain catalytic reagents that are dried to the interior surface of the chamber 1630, and those reagents are hydrolyzed and deployed into the analysis chamber 1642 to amplify the signal from the sensor, as described above in the embodiments of FIGS. 4 and 5. Applications of electrochemical techniques are described in further detail in U.S. Pat. Nos. 7,361,470 and 7,741,033, and PCT Application No. PCT/US12/024,015, which are hereby incorporated by reference herein in their entireties.

In particular, in preferred embodiments the reagents included in the reagent chamber 1630a are a redox pair having a first transition metal complex and a second transition metal complex, which together form an electrocatalytic reporter system (ECAT system) which amplifies the signal from the sensor, indicating a match between the genetic sequence fragments in the lysed sample and the sequences of the PNA probe. Examples of such pairs and amplification are Ru(NH₃)₆³⁺ and Fe(CN)₆³⁻, as further described in U.S. Provisional application No. 61/700,285. These reagents are dried down to the interior walls of the chamber 1630a. A blister 1631 contains a phosphate buffered salient solution (PBS) that is undiluted from a stock sample (thus the 1×). As will be explained below, after the sample buffer enters the tube 1602, the blister 1631 is punctured and flows into the chamber 1630b and thereafter mixes with the components of the ECAT system in 1630a to form a rehydrated reagent solution. The rehydrated reagent solution later flows into the analysis chamber 1642, where it meets with the lysate contents from the neutralization chamber 102b after they are bound and annealed to the sensor, as explained previously and further described below.

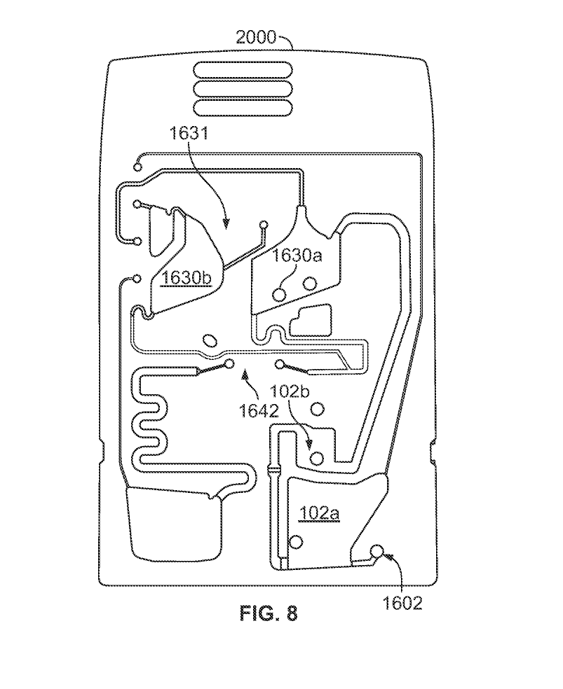

FIG. 8 depicts in further detail components of this hand-held system 2000, also referred to as a device 2000. As shown, the neutralization chamber 102b contains neutralization chemicals 103 (e.g., an acid) and the lysis chemical chamber 102a contains a lysis agent (e.g., a strong base such as NaOH). As explained above in regard to FIGS. 3A-4, the neutralization agent and lysis agents are preferably dried to the interior surface of their respective chambers 102b and 102a.

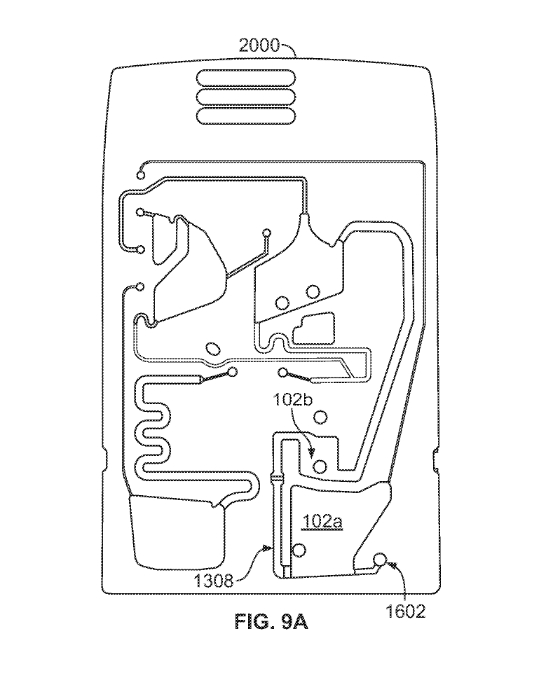

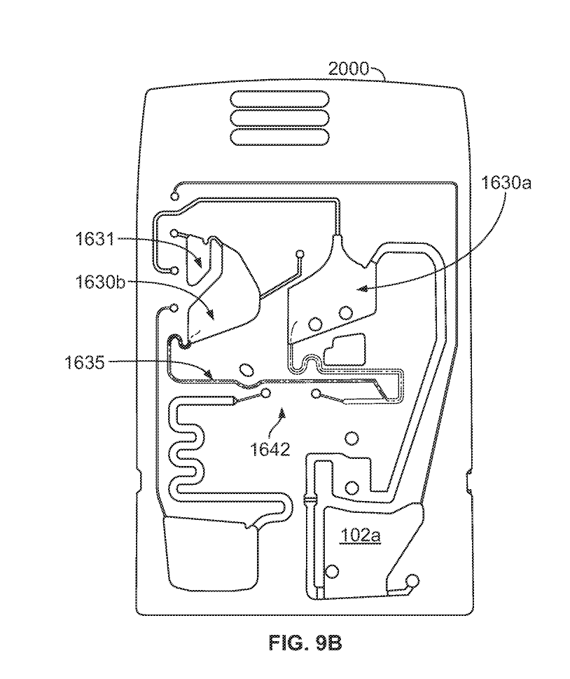

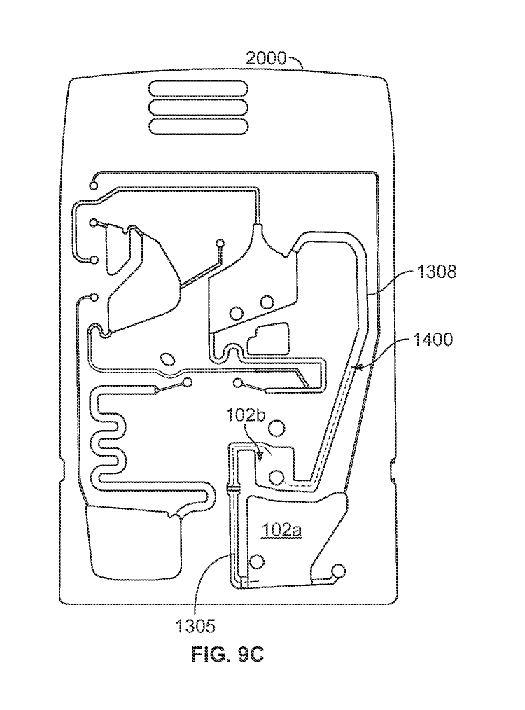

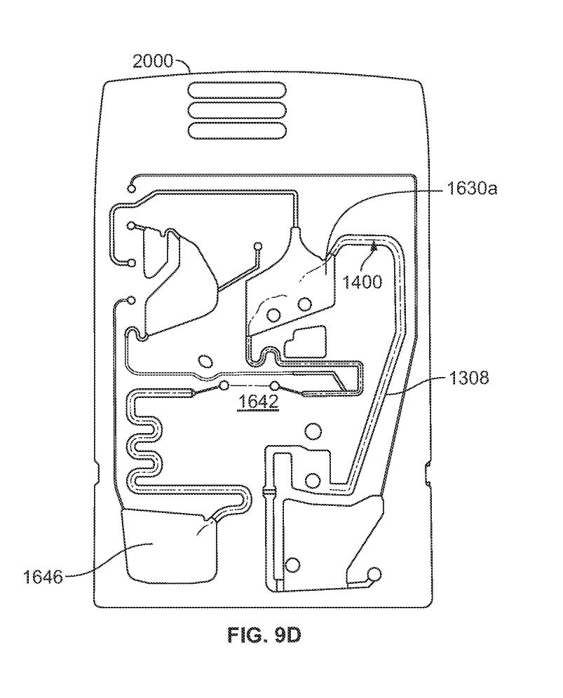

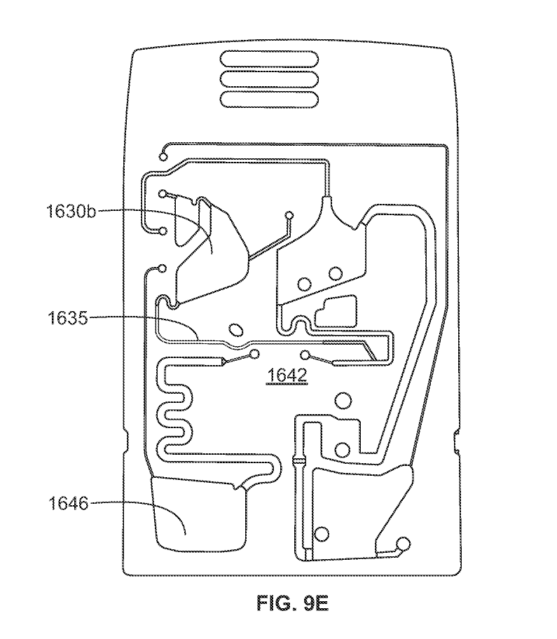

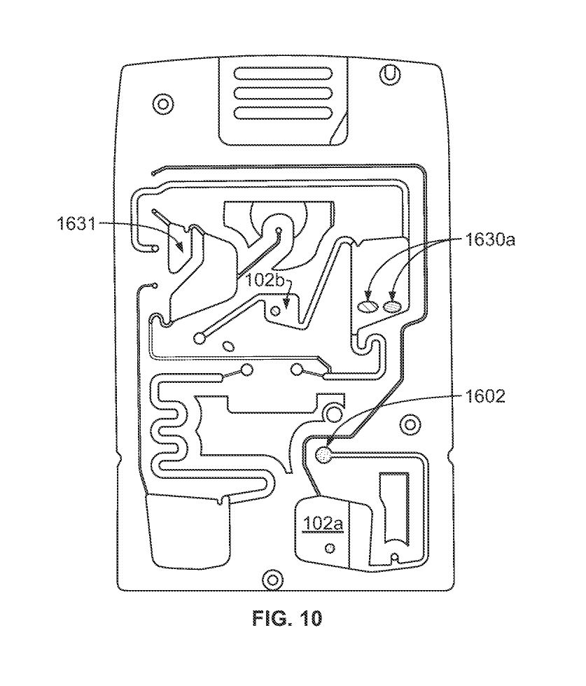

FIGS. 9A-9E depict the use and operation of the system 2000 or the hand-held device 2000. In a first step as shown in FIG. 9A, the sample is inserted into the sample chamber by the inlet port 1602 and flows by tube 1308 into the lysing compartment 102a. Inside the lysing compartment 102a, a strong lysing agent is provided, for example a base such as NaOH. The lysing agent is preferably dried to the interior surface of the compartment 102a. In certain implementations that agent may be dried within a well or separate receptacle located within the compartment 102a. In a second step, as shown in FIG. 9B, the blister 1631 is ruptured and releases the PBS into the metering chamber 1630b and is then pumped into the rehydrolysis chamber 1630a where the electrode catalytic agents (e.g., the ruthenium and ferric agents identified above) are located and preferably dried to the interior surface of the chamber 1630a. The chamber 1630a in this embodiment serves as a multi-use flow chamber to which it can both store the electrode catalytic agents and serve as the locale for rehydrating them, and also function as a receptacle for the receipt of the sample after it has lysed in the lysing chamber 1306, as described below.

After the blister 1631 has ruptured, the fluid in the blister flows into the metering chamber 1630b and is pumped through channel 1635 into the rehydration chamber 1630a whereupon it mixes with the catalytic agents which are dried to the interior surface of the chamber 1630a. The dried agents are solubilized in the blister fluid and thereafter they are pumped in reverse direction through channel 1635 back into the metering chamber 1630b, where they are stored for later use. Alternative designs could be used, where the solubilized electrocatalytic agents (e.g., the ECAT Ru and Fe components) are stored in the rehydration chamber 1630a and then applied directly to the sensor area 1642.

FIG. 9C depicts a next step (which could be applied in reverse order with the step of FIG. 9B). In this step the sample, which was lysed previously in the lysate formed in the chamber 102a, is pumped into the neutralization chamber 102b, where it dissolves a spot of dried neutralizing agent (such as an acid). As that dissolving occurs, the buffer flowing with the sample from chamber 102a is neutralized in its pH, achieving a pH that is less basic than the pH of the buffer while in chamber 102a. In preferred implementations the neutralizing agent in chamber 102b produces a solution of neutral pH such that the solution that exits the chamber 102b via flow outlet 1038 is of neutral pH and is ready for application to the sensor. That sample leaves the neutralization chamber via flow tube 1308 and is identified in FIG. 9C as sample 1400.

As shown in FIG. 9D, the sample 1400 which is preferably neutralized in its pH flows into the hydration chamber 1630a, which in this embodiment has a multi-purpose use for not only storing the catalytic agents for rehydration, but also then stores the neutralized and lysed sample solution 1400 prior to application to the sensor. This neutralized sample flows through the rehydration chamber 1630a and it slowly moved across the sensor 1642 where it is subject to the hybridization with the probe located in the sensor 1642 area. The neutralized sample flows down to the waste chamber 1646 after contacting the sensor area 1642. As depicted in FIG. 9E, after loading the sample onto the sensor 1642, the rehydrated electrocatalytic agents then flow slowly from the chamber 1630b through the flow channel 1635 and back to the sensor plate in area 1642. After the catalytic agents are applied to the sensor then analysis occurs as described above and as explained further in the U.S. Provisional Application No. 61/700,285, the contents of which are incorporated by reference. Applications of electrochemical analysis that can be used are also described in further detail in U.S. Pat. Nos. 7,361,470 and 7,741,033, and PCT Application No. PCT/US12/024,015, which are hereby incorporated by reference herein in their entireties.

FIG. 10 illustrates an example performed using the system 2000, including illustrative dried components and their concentrations used in the point of care system 2000. For example, the ECAT components are dried down separately in chamber 1630a with Ru(NH₃)₆³⁺ (30 μl at 0.017 mM) and Fe(CN)₆³⁻ (30 μl of 7.1 mM). Spots of those components are rehydrated with 213 μl of PBS, which is stored in blister 1631. The lysis sources (chemical agents) are dried to the chambers 102a and 102b. The lysing agent (NaOH in this example) is provided in a 10 μl dried spot on surface 102a. A sample buffer of 200 μl (0.2 M phosphate buffer at pH 7.2) containing CT bacterial cells is provided through the sample port 1602. Dissolution of the NaOH spot raises the buffer pH to pH 11 and lyses the bacteria in approximately 3 minutes. Lysis is stopped by neutralizing the buffer to pH 7.2 in chamber 102b, using Citric Acid. The Citric Acid (10 μl, of 1M) was dry spotted onto the interior surface of the chamber 102b.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. A chamber located within a point of care device, wherein the chamber includes at least one agent in dry form.

2. The chamber of claim 1, wherein the chamber is a lysis chamber disposed between an inlet port of the device and a probe positioned within the device, the lysis chamber having at least one chemical lysing agent disposed therein.

3. The chamber of claim 2, wherein the at least one chemical lysing agent is in dry form.

4. The chamber of claim 2, wherein the at least one chemical lysing agent is dried to an interior surface of the lysis chamber.

5. The chamber of any claim 1, wherein the lysis chamber includes first and second chambers and a flow line disposed between the first and second chambers and through which fluid flows from the first to the second chamber.

6. The chamber of claim 5, wherein the first chamber includes a chemical lysing agent and the second chamber includes a neutralizing agent.

7. The chamber of claim 6, wherein the chemical lysing agent is a base and the neutralizing agent is an acid.

8. The chamber of claim 6, wherein the chemical lysing agent is an acid and the neutralizing agent is a base.

9. The chamber of claim 6 or 7, wherein the base is NaOH.

10. The chamber of claim 5, wherein a base is dried to an interior surface of the first chamber and an acid is dried to an interior wall of the second chamber.

11. The chamber of claim 7, wherein a fluid sample includes cells containing genetic material and, upon the fluid sample's contacting the base in the first chamber, the fluid sample forms a lysate comprising lysed cells and fragments of the genetic material, the lysate having a basic pH.

12. The chamber of claim 11, wherein the fragments of the genetic material are partial fragments of the genetic material.

13. The chamber of claim 11, wherein the lysate flows out of the second chamber having a pH that is less basic than the pH of the lysate that exits the first chamber.

14. The chamber of claim 13, wherein the lysate flowing out of the second chamber has a neutral pH.

15. The chamber of claim 2, wherein the chemical lysing agent is mixed with a detergent.

16. The chamber of claim 1, comprising a catalytic agent dried to an interior surface of the chamber.

17. A point of care device having:
an inlet port through which a fluid sample flows,
a probe chamber, and;
a chamber according to any of the preceding claims.

18. The point of care device of claim 17, wherein the chamber is a lysis chamber.

19. The point of care device of claim 17, wherein the chamber is a catalytic agent chamber and contains one or more electrochemical agents configured to amplify an electrochemical signal arising from the device.

20. The device of claim 19, wherein the electrochemical agents include at least Ru(NH₃)₆³⁺ or Fe(CN)₆³⁻.

21. A method of preparing a biological sample for analysis of its nucleic acid material, comprising the steps of:
(i) combining the biological sample in a buffer to form a first solution at a first pH,
(ii) flowing the first solution into contact with a first chemical agent that changes the pH of the first solution, forming a second solution at a second pH, and;
(iii) flowing the second solution into contact with a second chemical agent that changes the pH to a level that is less basic or less acidic than the second pH.
